(19) United States
(12) Patent Application Publication
(10) Pub. No.: US 2009/0238766 A1
(43) Pub. Date: Sep. 24, 2009

(54) BIOLUMINESCENT CONSTRUCT

(75) Inventors: David J. Frost, Deerfield, IL (US); Jonathan A. Hickson, Lake Villa, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, ABBOTT PARK, IL (US)

(21) Appl. No.: 12/407,273

(22) Filed: Mar. 19, 2009

Related U.S. Application Data
(60) Provisional application No. 61/037,775, filed on Mar. 19, 2008.

(51) Int. Cl. A61K 49/00; C07K 5/00

(52) U.S. Cl. 424/9.6; 530/330

(57) ABSTRACT

The invention provides a construct comprising an amino acid sequence attached to a luciferin substrate, wherein the amino acid sequence is not a substrate for either luciferase or caspase-3/7 to render the substrate inactive in the presence of luciferase. The construct is useful, for example, as a negative control for in vitro apoptosis assays and the bioluminescent imaging of drug-induced apoptosis in vivo. The invention also provides methods for screening for novel modulators of an apoptosis-related disease.

### CROSS-REFERENCE SECTION TO RELATED APPLICATION

This application claims the benefit of U.S. Provisional Patent Application No. 61/037,775, filed Mar. 19, 2008, which is hereby incorporated by reference in its entirety.

### FIELD OF THE INVENTION

The invention provides a construct comprising an amino acid sequence attached to a luciferin substrate, wherein the amino acid sequence is not a substrate for either luciferase or a caspase to render the substrate inactive in the presence of luciferase. The construct is useful, for example, as a negative control for in vitro apoptosis assays and the bioluminescent imaging of drug-induced apoptosis in vivo. The invention also provides methods for screening for novel modulators of an apoptosis-related disease.

### BACKGROUND OF THE INVENTION

Optical imaging is a noninvasive technique that utilizes bioluminescent and fluorescent endogenous reporters or exogenous probes to monitor molecular and biological processes. While both modalities detect levels of visible light, a clear distinction exists between them with respect to how this light is created. Bioluminescence (e.g. firefly luciferase using the substrate luciferin) is the production and emission of light resulting from a chemical reaction during which chemical energy is converted to light energy (560-660 nm). This is an enzymatic process that requires a chemical substrate in order for light to be produced. Alternatively, fluorescence (e.g. GFP, RFP and near-IR proteins) occurs when the molecular absorption of a photon at one wavelength (400-600 nm) triggers the emission of another photon with a longer wavelength (450-650 nm). Fluorogenic compositions for the detection of protease activity are known, for example, in WO 98/37226.

Imaging of drug-induced apoptosis has become a focal interest in both experimental and clinical research. Z-DEVD-aminoluciferin (VivoGlo™ Caspase-3/7), manufactured by Promgea, is a substrate for luciferase only when cleaved by a cysteine aspartic acid-specific protease. Typically, the cysteine aspartic acid-specific proteases are cysteine aspartic acid-specific protease-3 (“caspase-3”) and cysteine aspartic acid-specific protease-7 (“caspase-7”), molecular markers of apoptosis. Methods of monitoring caspase-3/7 activity in vivo would provide a vaulable tool for assessing preclinical drug efficacy, allowing for quicker development of therapies. U.S. Pat. No. 7,148,030 describes a sensitive bioluminescent assay to detect proteases including caspases. Use of aminoluciferins for in vivo imaging is also known. Shah et al. (Molecular Therapy, Vol. 11, No. 6: 926-931) describes the single time-point use of a caspase-3-activatable aminoluciferins for in vivo bioluminescent imaging. One hurdle that has to be overcome with the use of aminoluciferase agents is the need for an appropriate negative control that is not a substrate for either luciferase or caspase-3/7.

### SUMMARY OF THE INVENTION

The invention provides a construct comprising an amino acid sequence attached to a luciferin substrate, wherein the amino acid sequence is not a substrate for either luciferase or a caspase, to serve as a negative control for a ZDEVD-aminoluciferin. Since the sequence is not a substrate for a caspase, even in apoptotic cells no light will be emitted. Thus, such a molecule would serve as a negative control for either in vitro apoptosis assays or for in vivo bioluminescent imaging studies utilizing, for instance, the VivoGlo™ Caspase-3/7 substrate.

In one embodiment, the present invention provides a construct comprising a sequence that is not a substrate for either luciferase or a caspase attached to a luciferin molecule. In another embodiment, the sequence is selected from the group consisting of DEVN, DDDD, FA, VEID, LEHD, and YVAD. In a more preferred embodiment, the sequence is DEVN. In another embodiment, the present invention comprises the construct as a negative control for an in vitro apoptosis assay. In another embodiment, the present invention comprises the construct as a negative control for in vivo bioluminescent imaging. In another embodiment, the luciferin molecule is an aminoluciferin. In one embodiment, the present invention comprises a method of identifying modulators of an apoptosis-related disease.

### DETAILED DESCRIPTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art.

All amino acid abbreviations used in this disclosure are those accepted by the United States Patent and Trademark Office as set forth in 37 C.F.R. sctn. 1.822(b).
“apoptosis-related disease”—a disease whose etiology is related either wholly or partially to the process of apoptosis. The disease may be caused either by a malfunction of the apoptotic process (such as in cancer or an autoimmune disease) or by overactivity of the apoptotic process (such as in certain neurodegenerative diseases).
“Cancer” or “Tumor”—an uncontrolled growing mass of abnormal cells. These terms include both primary tumors, which may be benign or malignant, as well as secondary tumors, or metastases which have spread to other sites in the body.
“Modulator”—any molecule that is capable of modulation, i.e. that either increases (promotes) or decreases (prevents). The term is understood to include partial or full inhibition, stimulation and enhancement.
“Treatment”—the term “treatment” and corresponding terms “treat” and “treating” includes palliative, restorative, and preventative treatment of a subject. The term “palliative treatment” refers to treatment that eases or reduces the effect or intensity of a condition in a subject without curing the condition. The term “preventative treatment” (and the corresponding term “prophylactic treatment” refers to treatment that prevents the occurrence of a condition in a subject. The term “restorative treatment” refers to treatment that halts the progression of, reduces the pathologic manifestations of, or entirely eliminates a condition in a subject.

### Embodiments of the Constructs

In one embodiment, the present invention comprises a construct comprising an amino acid sequence attached to a luciferin substrate, wherein the amino acid sequence is not a substrate for either luciferase or a caspase. Preferably, the caspase is caspase-3, caspase-7 or both caspase-3/7. Preferably, the sequence that is not a substrate for either luciferase or caspase-3/7 is four amino acids in length. More preferably, the sequence is four amino acids in length and has at least 25% homology to the sequence DEVD. More preferably, the sequence is four amino acids in length and has at least 50% homology to the sequence DEVD. Even more preferably, the sequence is four amino acids in length and has at least 75% homology to the sequence DEVD. In a preferred embodiment, the sequences are selected from the group consisting of DEVN, DDDD, FA, VEID, LEHD, and YVAD. In a more preferred embodiment, the sequences are selected from the group consisting of DEVN and LEHD. In a more preferred embodiment, the sequence is DEVN. In another embodiment, the present invention comprises the use of these sequences as a negative control for in vivo imaging.

In one embodiment, the luciferin molecule is selected from the group consisting of a bacterial luciferin, a dinoflagellate luciferin, a vargula luciferin, a coelenterasine luciferin, a firefly luciferin and a beetle luciferin. In another embodiment, the luciferin molecule is a beetle luciferin. In another embodiment, the luciferin molecule is an aminoluciferin. In a more preferred embodiment, the luciferin molecule is an aminoluciferin having the structure wherein the luciferin molecule is an amino luciferin having the structure

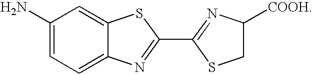

In another embodiment, the construct is selected from the group consisting of

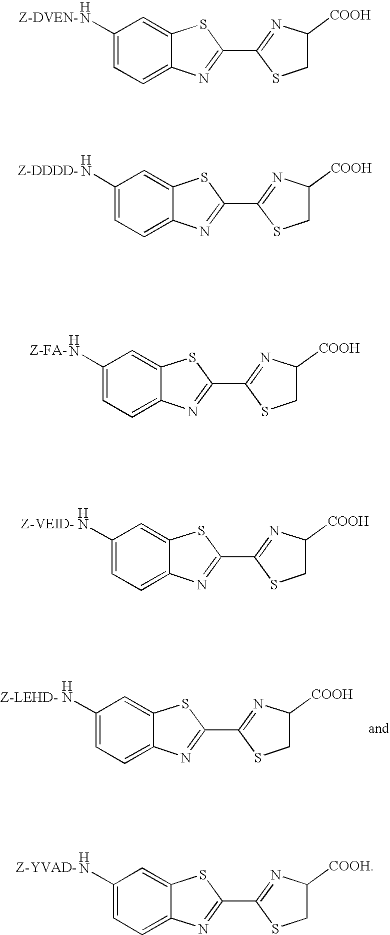

### Methods of Identifying Modulators of an Apoptosis-Related Disease

In one embodiment, the present invention comprises a method of identifying modulators of an apoptosis-related disease comprising
(a) administering a modulator of an apoptosis-related disease and a construct comprising a caspase-3/7 substrate attached to a luciferin molecule to luciferase-expressing apoptosis-related diseased cells in a first container;
(b) measuring the photon emission of the first container with a photodetector device;
(c) administering a modulator of an apoptosis-related disease and a construct comprising a sequence that is not a substrate for either luciferase or caspase-3/7 attached to a luciferin molecule to luciferase-expressing apoptosis-related diseased cells;
(d) measuring the photon emission of the second container with a photodetector device; and
(e) comparing the photon emission of the first container and the second container.

A modulator of an apopotosis-related disease, wherein the modulator is pro-apoptotic, will have increased the photon emission of the first container. A modulator of an apopotosis-related disease, wherein the modulator is anti-apoptotic, will have decreased the photon emission of the first container. It is understood that it is within the scope of the method for the first measure and the second measure to be taken in the reverse chronological order. The container can be, for instance, a well in a microtiter plate. In one embodiment, the plate is a 96-well plate. In another embodiment, the construct of step (c) is selected from the group consisting of DEVN, DDDD, FA, VEID, LEHD, and YVAD and the luciferin molecule is an amino luciferin having the structure

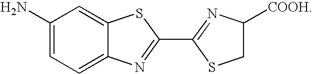

In one embodiment of the methods of the present invention, the subject is a mammal. In another embodiment, the subject is a rat. In another embodiment, the subject is a mouse. In another embodiment, the subject is a human.

In one embodiment, the present invention comprises a method of identifying modulators of an apoptosis-related disease comprising
(f) administering to a first subject luciferase-expressing cells;
(g) allowing the cells to achieve localization in the first subject;
(h) administering a modulator of an apoptosis-related disease and a construct comprising a caspase-3/7 substrate attached to a luciferin molecule to the first subject;
(i) imaging the photon emission of the first subject with a photodetector device;
(j) administering to a second subject luciferase-expressing cells;
(k) allowing the cells to achieve localization in the second subject;
(l) administering a modulator of an apoptosis-related disease and a construct comprising a sequence that is not a substrate for either luciferase or caspase-3/7 attached to a luciferin molecule to the second subject;
(m) imaging the photon emission of the second subject with a photodetector device; and
(n) comparing the photon emission of the first subject and the second subject.

A modulator of an apopotosis-related disease, wherein the modulator is pro-apoptotic, will have increased the photon emission of the first subject. A modulator of an apopotosis-related disease, wherein the modulator is anti-apoptotic, will have decreased the photon emission of the first subject. It is understood that it is within the scope of the method for the first image and the second image to be taken in the reverse chronological order. In another embodiment, the construct of step (1) is selected from the group consisting of DEVN, DDDD, FA, VEID, LEHD, and YVAD and the luciferin molecule is an amino luciferin having the structure

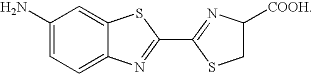

In another embodiment, the present invention comprises a method of identifying modulators of an apoptosis-related disease comprising
(a) administering to a subject luciferase-expressing cells;
(b) allowing the cells to achieve localization in the subject;
(c) administering a modulator of an apoptosis-related disease and a construct comprising a caspase-3/7 substrate attached to a luciferin molecule to the subject;
(d) creating a first image of the photon emission of the subject with a photodetector device;
(e) administering a modulator of an apoptosis-related disease and a construct comprising a sequence that is not a substrate for either luciferase or caspase-3/7 attached to a luciferin molecule to the subject;
(f) creating a second image of the photon emission of the subject with a photodetector device; and
(g) comparing the photon emission of the first image and the second image.
   A modulator of an apopotosis-related disease, wherein the modulator is pro-apoptotic, will have increased the photon emission of the first image. A modulator of an apopotosis-related disease, wherein the modulator is anti-apoptotic, will have decreased the photon emission of the second image. In another embodiment, the construct of step (e) is selected from the group consisting of DEVN, DDDD, FA, VEID, LEHD, and YVAD and the luciferin molecule is an amino luciferin having the structure

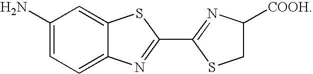

In another embodiment, the present invention comprises a method of identifying modulators of an apoptosis-related disease comprising
(a) administering to a subject luciferase-expressing cells;
(b) allowing the cells to achieve localization in the subject;
(c) administering a modulator of an apoptosis-related disease and a construct comprising a sequence that is not a substrate for either luciferase or caspase-3/7 attached to a luciferin molecule to the subject;
(d) creating a first image of the photon emission of the subject with a photodetector device;
(e) administering a modulator of an apoptosis-related disease and a construct
(f) comprising a caspase-3/7 substrate attached to a luciferin molecule to the subject;
(g) creating a second image of the photon emission of the subject with a photodetector device; and
(h) comparing the photon emission of the first image and the second image.

A modulator of an apopotosis-related disease, wherein the modulator is pro-apoptotic, will have increased the photon emission of the second image. A modulator of an apopotosis-related disease, wherein the modulator is anti-apoptotic, will have decreased the photon emission of the first image.

In another embodiment of the methods of identifying modulators of an apoptosis-related disease, the construct comprising a sequence that is not a substrate for either luciferase or caspase-3/7 attached to a luciferin molecule comprises a sequence selected from the group consisting of DEVN, DDDD, FA, VEID, LEHD, and YVAD. In another embodiment of the methods of identifying modulators of an apoptosis-related disease, the construct comprising a sequence that is not a substrate for either luciferase or caspase-3/7 attached to a luciferin molecule comprises DEVN. In another embodiment of the methods of identifying modulators of an apoptosis-related disease, the construct comprising a sequence that is not a substrate for either luciferase or caspase-3/7 attached to a luciferin molecule, wherein the luciferin molecule is an amino luciferin having the structure

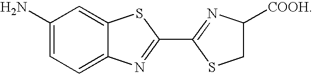

In another embodiment of the methods of identifying modulators of an apoptosis-related disease, the construct comprising a sequence that is not a substrate for either luciferase or caspase-3/7 attached to a luciferin molecule comprises a sequence selected from the group consisting of DEVN, DDDD, FA, VEID, LEHD, and YVAD and the luciferin molecule is an amino luciferin having the structure

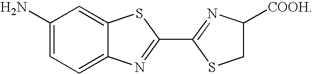

In another embodiment of the methods of identifying modulators of an apoptosis-related disease, the construct comprising a sequence that is not a substrate for either luciferase or caspase-3/7 attached to a luciferin molecule is DEVN and the luciferin molecule is an amino luciferin having the structure

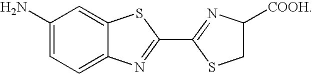

In another embodiment, the apoptosis-related disease is a proliferative disease selected from the group consisting of a tumor disease (including benign or cancerous) and/or any metastases, wherever the tumor or the metastasis are located, including breast cancer including, for example, advanced breast cancer, stage 1V breast cancer, locally advanced breast cancer, and metastatic breast cancer, lung cancer, including, for example, non-small cell lung cancer (NSCLC, such as advanced NSCLC), small cell lung cancer (SCLC, such as advanced SCLC), and advanced solid tumor malignancy in the lung; ovarian cancer, head and neck cancer, gastric malignancies, melanoma (including metastatic melanoma), colorectal cancer, pancreatic cancer, and solid tumors (such as advanced solid tumors); hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. In another embodiment, the apoptosis-related disease is selected from the group consisting of ocular diseases such as cataracts or glaucoma, osteoporosis, bone fractures, epidermal lesions, restenosis, conditions linked to an incorrect proliferation or migration of the smooth muscle cells, inflammations of the respiratory system, asbestosis, silicosis, lupus erythematosus, Goodpasture's syndrome, granulomatosis, eosinophilic granulomatosis, gastric and duodenal ulcers, oesophagitis, enteritis, gastritis, septicaemia, dysfunctions of the haematopoiesis and/or lymphopoiesis, cystic fibrosis, myelopathies and arthropathies, hepatites (C, A, B, F), AIDS, immune deficiencies, cell aging, tissue degeneration phenomena, inflammation, infectious diseases, graft rejection, acute or chronic rheumatoid arthritis, ulcerative colitis, thrombocytopenic purpura, autoimmune erythronoclastic anaemia, juvenile (Type I) diabetes (insulin-dependent), myelodysplasic syndrome, Huntington's disease, prion diseases, ARDS, prostatic hypertrophy, asthma, atherosclerosis and its thrombo-embolic complications, renal diseases, glomerulonephritis, ischemic pathologies such as myocardial infarction, myocardial ischemia, coronary vasospasm, angina and cardiac failure, chronic pancreatitis, auto-immune gastritis, and primary biliary cirrhosis.

In another embodiment, the present invention comprises a modulator of an apoptosis-related disease identified by the methods of identifying modulators of an apoptosis-related disease. In another embodiment, the modulator is an anti-apoptotic drug. In another embodiment, the modulator is an anti-tumor drug. In another embodiment, the method is used to identify an appropriate efficacious dose of the modulator of an apoptosis-related disease.

In one embodiment, the localization of the luciferase-expressing cells takes from about 1 minute to about 1 hour. In another embodiment, the localization of the luciferase-expressing cells takes from about 5 minutes to about 30 minutes.

In one embodiment, the present invention comprises a method of evaluating the efficacy of modulators of an apoptosis-related disease comprising
(a) administering to a first subject luciferase-expressing cells;
(b) allowing the cells to achieve localization in the first subject;
(c) administering a modulator of an apoptosis-related disease and a construct comprising a caspase-3/7 substrate attached to a luciferin molecule to the first subject;
(d) imaging the photon emission of the first subject with a photodetector device;
(e) administering to a second subject luciferase-expressing cells;
(f) allowing the cells to achieve localization in the second subject;
(g) administering a modulator of an apoptosis-related disease and a construct comprising a sequence that is not a substrate for either luciferase or caspase-3/7 attached to a luciferin molecule to the second subject;
(h) imaging the photon emission of the second subject with a photodetector device; and
(i) comparing the photon emission of the first subject and the second subject.
   In another embodiment, the construct of step (g) is selected from the group consisting of DEVN, DDDD, FA, VEID, LEHD, and YVAD and the luciferin molecule is an amino luciferin having the structure

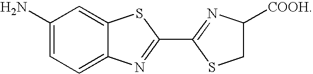

### Experimentals

Compounds of the invention can be prepared using procedures that are generally known. For example, compounds of the invention can be prepared using standard solution phase chemistry.

### Synthesis of Caspase-3/7 Inactive Substrate(s):

Compounds of the invention can be prepared using conventional peptide synthesis protocols. Similar chemical syntheses could be performed for the other inactive substrates.

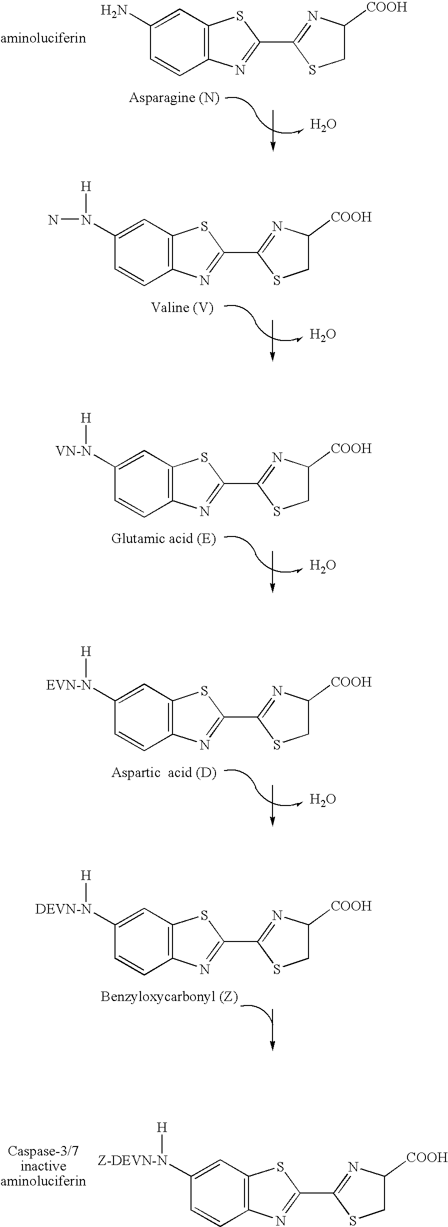

### In Vivo Bioluminescent Imaging of VivoGlo™ Caspase-3/7 Substrate and Caspase-3/7 Inactive Substrate(s):

To test for the sensitivity and specificity of the caspase-3/7 inactive substrates in vivo, mice bearing tumors stably expressing luciferase will be treated with known apoptosis-inducing cytotoxic therapies (e.g. docetaxel). At timepoints post treatment, mice will be injected intraperitoneally with 62.5 mg/mL of VivoGlo™ Caspase-3/7 substrate or caspase-3/7 inactive substrate(s). A buffer comprising about 30% PEG 400, about 5% DMSO, about 5% Tween 80 and about 60% Dextrose 5% in Water (D5W) is used for the VivoGlo™ Caspase-3/7 substrate and caspase-3/7 inactive substrate(s). In luciferase-expressing cells undergoing apoptosis, caspase-3/7 will cleave the DEVD peptide from the VivoGlo™ Caspase-3/7 substrate, allowing it to be a substrate for luciferase photon production. In luciferase-expressing cells undergoing apoptosis, caspase-3/7 will be unable to cleave the peptide from the caspase-3/7 inactive substrate(s), preventing the substrate from being used for luciferase photon production. The photon emission will be detected with a photodetector device at timepoints from 5-30 minutes post injection and quantified. The VivoGlo Caspase-3/7 injected mice should yield significant bioluminescent signal, whereas the caspase-3/7 inactive substrate-injected mice should produce little to no bioluminescent signal, indicating specificity of the substrate.

We claim:

1. A construct comprising a sequence that is not a substrate for either luciferase or caspase attached to a luciferin molecule.

2. The construct of claim 1, wherein the sequence is four amino acids in length and has at least 25% homology to the sequence DEVD.

3. The construct of claim 1, wherein the sequence is four amino acids in length and has at least 50% homology to the sequence DEVD.

4. The construct of claim 1, wherein the sequence is four amino acids in length and has at least 75% homology to the sequence DEVD.

5. The construct of claim 1, wherein the sequence is selected from the group consisting of DEVN, DDDD, FA, VEID, LEHD, and YVAD.

6. The construct of claim 5, wherein the sequence is DEVN.

7. The construct of claim 1, wherein the luciferin molecule is a beetle luciferin.

8. The construct of claim 5, wherein the luciferin molecule is an amino luciferin having the structure

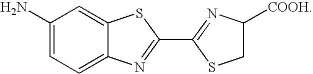

9. The construct of claim 8, wherein the construct is a negative control for an in vitro apoptosis assay.

10. The construct of claim 8, wherein the construct is a negative control for in vivo bioluminescent imaging.

11. A method of identifying a modulator of an apoptosis-related disease comprising
(a) administering to a first subject luciferase-expressing cells;
(b) allowing the cells to achieve localization in the first subject;
(c) administering a modulator of an apoptosis-related disease and a construct comprising a caspase-3/7 substrate attached to a luciferin molecule to the first subject;
(d) imaging the photon emission of the first subject with a photodetector device;
(e) administering to a second subject luciferase-expressing cells;
(f) allowing the cells to achieve localization in the second subject;
(g) administering a modulator of an apoptosis-related disease and a construct comprising a sequence that is not a substrate for either luciferase or caspase-3/7 attached to a luciferin molecule to the second subject;
(h) imaging the photon emission of the second subject with a photodetector device; and
(i) comparing the photon emission of the first subject and the second subject.

12. A method of identifying modulators of an apoptosis-related disease comprising
(a) administering to a subject luciferase-expressing cells;
(b) allowing the cells to achieve localization in the subject;
(c) administering a modulator of an apoptosis-related disease and a construct comprising a caspase-3/7 substrate attached to a luciferin molecule to the subject;
(d) creating a first image of the photon emission of the subject with a photodetector device;
(e) administering a modulator of an apoptosis-related disease and a construct comprising a sequence that is not a substrate for either luciferase or caspase-3/7 attached to a luciferin molecule to the subject;
(f) creating a second image of the photon emission of the subject with a photodetector device; and
(g) comparing the photon emission of the first image and the second image.

13. A method of identifying modulators of an apoptosis-related disease comprising
(i) administering to a subject luciferase-expressing cells;
(j) allowing the cells to achieve localization in the subject;
(k) administering a modulator of an apoptosis-related disease and a construct comprising a sequence that is not a substrate for either luciferase or caspase-3/7 attached to a luciferin molecule to the subject;
(l) creating a first image of the photon emission of the subject with a photodetector device;
(m) administering a modulator of an apoptosis-related disease and a construct
(n) comprising a caspase-3/7 substrate attached to a luciferin molecule to the subject;
(o) creating a second image of the photon emission of the subject with a photodetector device; and
(p) comparing the photon emission of the first image and the second image.

14. The method of claim 11 wherein the construct comprising a sequence that is not a substrate for either luciferase or caspase-3/7 attached to a luciferin molecule comprises a sequence selected from the group consisting of DEVN, DDDD, FA, VEID, LEHD, and YVAD.

15. The method of claim 11, wherein the construct comprising a sequence that is not a substrate for either luciferase or caspase-3/7 attached to a luciferin molecule comprises DEVN.

16. The method of claim 14, wherein the construct comprising a sequence that is not a substrate for either luciferase or caspase-3/7 attached to a luciferin molecule, wherein the luciferin molecule is an amino luciferin having the structure [chemical structure]
